(19) United States
(12) Patent Application Publication
(10) Pub. No.: US 2009/0156904 A1
(43) Pub. Date: Jun. 18, 2009

(54) Human Physiological Symptom Detecting and Processing Device Incorporated Into Transportation or Moving Facility

(76) Inventor: Ein-Yiao Shen, Taipei City (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 11/955,220

(22) Filed: Dec. 12, 2007

(51) Int. Cl. A61B 5/00

(52) U.S. Cl. 600/300

(57) ABSTRACT

A human physiological symptom detecting and processing device incorporated into transportation or moving facility comprising two touch and press strips and a detecting and processing units. The former are used to detect the operator's physiological symptoms/signs by touch and press action and send related information to the latter with a signal. The detecting and processing unit converts this signal to individual signals decipherable to a display terminal for displaying and alarming, and optionally to a communication device for transmitting the data to a remote place, and further optionally to a GPS for positioning. The device can detect the physiological condition of the operator(s) of the transportation or moving facility in a timely manner to minimize potential for accidents.

### BACKGROUND OF THE INVENTION

1. Field of the Invention

The present invention relates to human physiological symptom detecting and processing device incorporated into transportation or moving facility with which the operator of the facility can check up his/her own physiological symptoms any time and anywhere so as to ensure security in operating the facility, and to bring an extra value to the facility.

2. Description of the Prior Art

Keeping in pace with the progress of modern science, the transportation facilities, for example, automobiles, ships, aircrafts and the like have achieved remarkable developments. When a user intends to purchase a new transportation equipment, the major considerations are not only its price, function, comfortability, stability and practicability, but also security. Accordingly, designers and manufacturers of transportation facilities should certainly lay emphasis on improving security in such facilities.

Taking the automobile for example. In addition to construct a firm and reliable vehicle body, there are a lot of safety accessories available such as a safety air bag, the ABS brake system, a back-up radar, and a third brake light, etc. Both software and hardware have progressively been improving to assure the user's safety. However, the aforesaid establishment is passive in nature, and what is more important is to monitor the driver's physiological conditions at all time. And as soon as any abnormal condition is detected, it can be remedied in time so as to assure traffic safety.

At present, the on-skin measurement of physiological data such as body temperature, blood pressure, heart-rhythm, blood sugar, oxygen supply to the heart, etc. is widely practicable, and even more advanced physiological data sensing device has become a matured technology. Therefore, it is desirous to develop an apparatus to process data obtained from a sensor, and display, memorize, store or even transmit via a signal to a desired destination.

Based on the above point of view, to entrain a human physiological symptom detecting and processing device on a transportation or a moving facility to monitor the operator's physiological symptoms/conditions any time and anywhere so as to prevent possible accident can be achieved and is worthy for consideration.

The inventor of the present invention has previously disclosed related information in Taiwan, namely, 1: “Physiological Condition Sensing Device Incorporated with Portable Phone” (Taiwan Pat. No. 004502). 2: Life Symbol Sensor Incorporated with Transmission Interface of Personal Digital Assistant” (Taiwan Pat. No. 00482000). 3: “Human Body Physiological Signal Treatment Device Incorporated with Portable Phone” (Taiwan Pat. No. 200412777). 4: “Earphone Life Symbol Detector” (Taiwan Pat. No. M241002). 5: “Life Information Wireless Transmission System” (Taiwan Pat. No. 1249122). 6: “Portable Communication Device with Ear Temperature Sensor”. (Taiwan Pat. No. M292221). 7: Ear Temperature Senso Incorporated in Portable Communication Device” (Taiwan Pat. No. M296028). 8: “Human Physiological condition Sensing Module in Portable Communication Device” (Taiwan Pat. No. M309299), all of which are herein incorporated by reference in their entireties.

However, there remains a need for for even further advanced improvement for the inventions enumerated above. All referenced patents, applications and literatures are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. The invention may seek to satisfy one or more of the above-mentioned desire. Although the present invention may obviate one or more of the above-mentioned desires, it should be understood that some aspects of the invention might not necessarily obviate them.

### SUMMARY OF THE INVENTION

An object of the present invention is to provide a human physiological symptom detecting and processing device incorporated into a transportation or moving facility with which the operator or operators of the transportation or moving facility can monitor his/her own physiological symptoms/conditions any time and anywhere, so as to assure traffic safety and to also offer the facility an extra value.

Another object of the present invention is to provide a human physiological symptom detecting and processing device to incorporate into a transportation or moving facilities such as aircrafts, ships including submarines, automobiles, rockets and space shuttles so as to detect and process the physiological data of the operator or operators simply by touch and press.

Still another object of the present invention is to provide aforesaid device with relative low cost, but widely applicable and easily operable.

To achieve these and other objects described above, the device of the present invention comprises a pair of touch and press strips and a detecting and processing unit. The former are used to pick up the operator's physiological symptoms by touch and press action, and send the related information to the latter with a signal. The detecting and processing unit converts the physiological signal to individual signals decipherable to a display terminal for displaying and alarming, to a communication device for transmitting the data to a remote place, and to Global Positioning System (GPS) for positional information. The display terminal, the communication device, and the correspondent device with GPS are disposed at a proper place in the transportation or moving facility such that the device of the present invention is able to monitor the physiological condition of the operator(s) thereby preventing occurrence of an accident.

### BRIEF DESCRIPTION OF THE DRAWINGS

The present invention may best be understood through the following description with reference to the accompanying drawings, in which:

FIG. 1 is a block diagram of the human physiological symptom detecting and processing device incorporated into transportation and moving facilities according to the present invention; and

FIG. 2 and FIG. 3 are respectively a first and a second embodiments of the present invention.

### DETAILED DESCRIPTION OF THE PREFERRED EMBODIMENTS

As used herein, the term “facility” in conjunction with transportation refers to a vehicle, a craft, a machinery, or other human-operated devices.

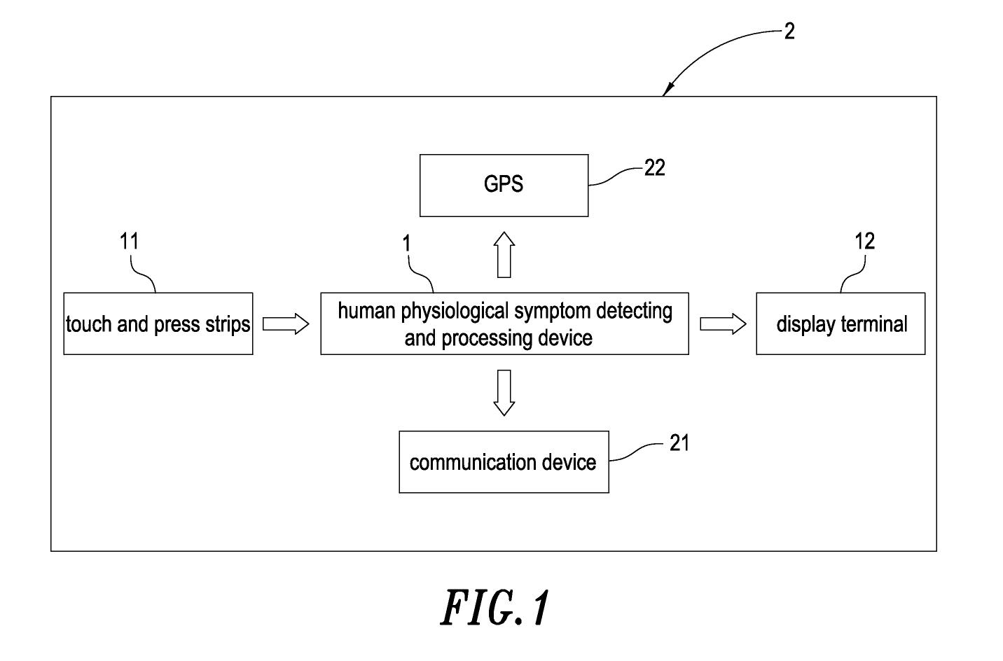

Referring to FIG. 1, in this block diagram of the present invention, a human physiological symptom detecting and processing device 1 is disposed inside of a transportation or a moving facility 2, and the device 1 is composed of a pair of touch and press strips 11 and a detecting and processing unit.

The touch and press strip 11 is used to sense human physiological symptoms/signs such as electro-activity in the heart, heart-rhythm, blood sugar level, blood pressure, blood oxygen, electro-activity in the brain, body temperature, aspiration rate, etc. and output the data to the detecting and processing unit. The touch and press strip 11 is made of an electrically conductive strip attached to the operation handle or rod of the transportation or moving facility 2, or disposed in the gloves of the operator(s) to deliver a sensed signal when being pressed.

The detecting and processing unit of the device 1 processes the data signal sent from the touch and press strips 11 and converts it to an individually decipherable signals, and outputs to a display terminal 12 and a communication device 21. The operation of the human physiological symptom detecting and processing device 1 is as follows:

1.Detecting and recording the electrocardiac phenomenon of Lead 1 and heart-rhythm to establish a cardiogram, and monitoring ST segment to detect diminished oxygen supply to the heart early. The range of detection covers from Lead II, m ,AVR, AVF, AVL to V1˜V6.

2.Identifying finger prints.

3.Detecting the physiological condition by variation of the touching pressure.

4.Detecting physiological symptoms such as blood sugar (non-invasive), blood pressure (optional), blood oxygen, body temperature, sweat, aspiration, electroencephalogram etc.

5.Incorporating with RFID.

6.Recording image and voice.

7.Preparing physiological monitoring record black box for the pilot or driver of the transportation facility.

The display terminal 12 is disposed at a position inside of the transportation or moving facility 2 to receive the decipherable signal of the physiological symptoms transmitted from the detecting and processing unit for displaying and alarming.

The communication device 21 receives the decipherable signal of the physiological symptoms transmitted from the detecting and processing unit and delivers a related signal to the remote destination.

A device 22 communicative with GPS can be provided to receive the decipherable signal of the physiological symptoms transmitted for the detecting and processing unit for communication with GPS to obtain a positioning information from GPS and delivering a related signal to a remote destination.

In the present invention, an electrical circuit is formed by contact of the human body with the touch and press strips 11 to detect the human body physiological symptoms which are sent and processed in the detecting and processing device 1 where it is converted to individually decipherable signals to optionally transmit to the display terminal 12 for displaying and alarming, and optionally to the communication device 21 for transmitting the information to a remote destination, or to the GPS device 22 to be positioned such that the operator's physiological condition can be notified and controlled remotely.

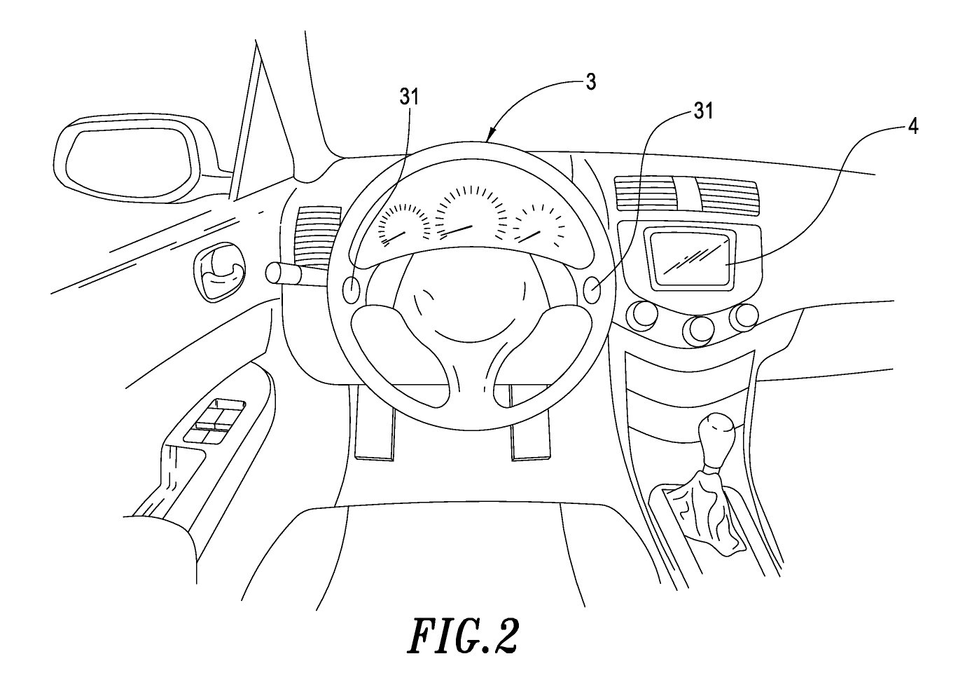

In an embodiment of the present invention shown in FIG. 2, wherein two electrically conductive strips 31 are disposed each at one side of the steering wheel 3 of the automobile. The driver can easily touch and press these two strips 31 with two thumbs to form an electrical circuit. The physiological symptom obtained thereof are detected and processed in the detecting and processing device 1 and converted to a decipherable signal output for a display terminal 4 equipped inside of the automobile for displaying or alarming. Or, the signal is outputted to a communication device and further transmitted to a remote destination, or the signal may be sent to GPS for positioning. In this manner, both the driver himself/herself or the remote monitor is able to watch the physiological condition of the driver constantly so as to avoid occurrence of traffic accident. In addition, an alarm may be installed at a marked location on the automobile to warn tailgating of vehicles, if necessary.

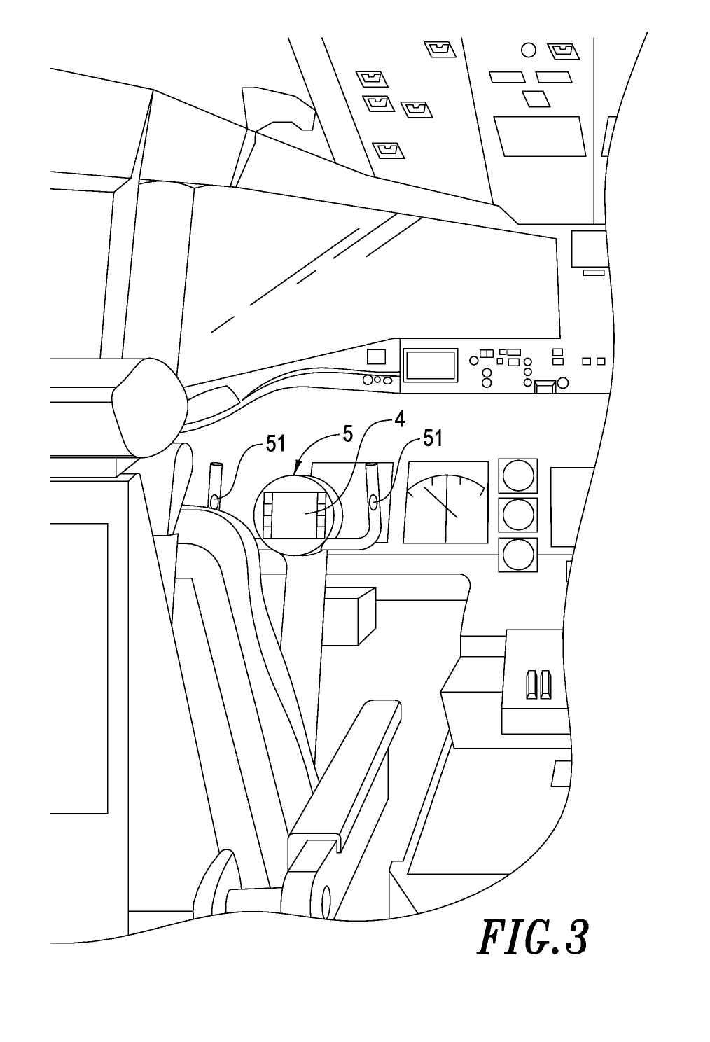

In another embodiment of the present invention shown in FIG. 3, as in an aircraft. In particular, an airforce pilot may often suffer from the sudden oppression of physiological or psychological pressure that can possibly lead to cardiac arrest or heart-rhythm disturbances. For this reason, as shown in FIG. 3, two electrically conductive strips 51 are employed to attach to the operation rod each at one side allowing the pilot to press them easily by thumbs. The electrical circuit formed as such may detect the physiological symptoms/vital signs of the pilot. The data signal is detected and processed in the detecting and processing device 1 to convert to a decipherable signal output for a display terminal 4, which is equipped inside the aircraft for displaying or alarming. Optionally, the signal can be transmitted to a communication device for further transmission to a remote destination. The signal may also be sent to via a GPS for positioning. In this manner, both the pilot and the remote monitor are able to watch the physiological condition of the pilot constantly so as to avoid unfortunate occurrence of air crash. Such physiological data obtained in a regular flight may also be utilized as a referential information in the flight personnel training course.

In short, it emerges from the description of the above embodiments that the invention has several noteworthy advantages, in particular,

1.As the detecting and processing device is incorporated into the transportation or moving facility, the driver or pilot is able to get his/her own physiological data any time and anywhere so as to avoid occurrence of possible accident.

2.The detecting and processing device is conveniently applicable to any types of transportation and moving facilities.

3.The device can perform multiple functions, such as displaying, alarming, remote transmitting, and even GPS positioning, while the device can be made compact, handy, and easily operable with at relative low cost.

Although the invention has been disclosed and illustrated with respect to preferred embodiments thereof, it is to be understood that the invention is not to be so limited and that other changes and modifications can be made within the full intended scope of the invention as hereinafter claimed.

What is claimed is:

1. Human physiological symptom detecting and processing device incorporated into transportation or moving facility comprising:
two touch and press strips for sensing human physiological symptoms and sending a signal to a detecting and processing unit; and
said detecting and processing unit for receiving said signal from said touch and press strips and converting it to individually decipherable signals to output to a display terminal, a communication device, and GPS.

2. Human physiological symptom detecting and processing device incorporated into transportation or moving facility comprising:
two touch and press strips for sensing human physiological symptoms and sending a signal to a detecting and processing unit;
said detecting and processing unit for receiving said signal from said touch and press strips and converting it to individually decipherable signals to output to a display terminal, a communication device, and GPS; and
a physiological symptom monitoring and controlling means disposed at a proper place inside of said transportation or moving facility and composed of said display terminal for receiving said deciphered signal to display and alarm, said communication device for receiving said deciphered signal to transmit to a remote destination, and a device receivable of GPS information for positioning.

3. The device of any of the claims from 1, wherein said touch and press strips are electrically conductive stripswhich form an electrical circuit when being pressed.

4. The device of any of the claims from 2, wherein said touch and press strips are electrically conductive stripswhich form an electrical circuit when being pressed.

5. The device of claim 3, wherein said touch and press strips are attached to the operational handle or rod of said transportation or moving facility.

6. The device of claim 4, wherein said touch and press strips are attached to the operational handle or rod of said transportation or moving facility.

7. The device of claim 3, wherein said touch and press strips are installed in the operator's gloves.

8. The device of claim 4, wherein said touch and press strips are installed in the operator's gloves.

9. The device of any of the claims from 1, wherein said detecting and processing device is able to detect and record the electrocardiac phenomenon of Lead 1 and heart-rhythm to establish a cardiogram, and also able to monitor ST segment to detect diminished oxygen supply to the heart early.

10. The device of any of the claims from 2, wherein said detecting and processing device is able to detect and record the electrocardiac phenomenon of Lead 1 and heart-rhythm to establish a cardiogram, and also able to monitor ST segment to detect diminished oxygen supply to the heart early.

11. The device of any of the claims from 1, wherein the detecting and processing range of said device covers from Lead II, III,AVR, AVF, AVL to V1˜V6 for achieving further effect.

12. The device of any of the claims from 2, wherein the detecting and processing range of said device covers from Lead II, III,AVR, AVF, AVL to V1˜V6 for achieving further effect.

13. The device of any of the claims from 1, wherein said device is able to identify human finger prints.

14. The device of any of the claims from 2, wherein said device is able to identify human finger prints.

15. The device of any of the claims from 1, wherein said device is able to detect and record data with pressing action.

16. The device of any of the claims from 2, wherein said device is able to detect and record data with pressing action.

17. The device of any of the claims from 1, wherein said device is able to detect blood sugar, blood pressure, blood oxygen, body temperature, sweat, aspiration and electroencephalogram.

18. The device of any of the claims from 2, wherein said device is able to detect blood sugar, blood pressure, blood oxygen, body temperature, sweat, aspiration and electroencephalogram.

19. The device of any of the claims from 1, wherein said device is able to record image and voice.

20. The device of any of the claims from 2, wherein said device is able to record image and voice.

21. The device of any of the claims from 1, wherein said device is able to record and store the physiological symptoms of said transportation or moving facity operator for further study.

22. The device of any of the claims from 2, wherein said device is able to record and store the physiological symptoms of said transportation or moving facity operator for further study.

23. The device of any of the claims from 1, wherein an alarm is installed at a marked location on the automobile to warn the tailing vehicles to avoid traffic accident.

24. The device of any of the claims from 2, wherein an alarm is installed at a marked location on the automobile to warn the tailing vehicles to avoid traffic accident.
